(19) United States
(12) Patent Application Publication
(10) Pub. No.: US 2024/0050916 A1
(43) Pub. Date: Feb. 15, 2024

(54) BIODEGRADABLE MICROCAPSULES BASED ON COMPOSITE MATERIAL AND SYNTHESIS PROCESS

(71) Applicant: MIKROCAPS D.O.O., Ljubljana (SI)

(72) Inventors: Aljosa Vrhunec, Ljubljana (SI); Dejan Stefanec, Dobrova (SI); Tomaz Kotnik, Podvleka (SI); Domen Kranjc, Kamnik (SI)

(21) Appl. No.: 18/267,442

(22) PCT Filed: Jan. 11, 2021
(86) PCT No.: PCT/SI2021/050001

§ 371 Date: Jun. 14, 2023

(30) Foreign Application Priority Data
Dec. 14, 2020 (SI) P-202000236

(51) Int. Cl. B01J 13/08; B01J 13/16

(52) U.S. Cl. CPC B01J 13/08; B01J 13/16

(57) ABSTRACT

The present invention relates to biodegradable microcapsules with a membrane formed of a biodegradable composite material and their synthesis. Biodegradable microcapsules obtained as described in the present invention consist of a core material comprising at least one liquid water-immiscible active component, and a membrane that encloses this core material, the membrane consisting of a composite material comprising a carrier polymer framework and at least one filler deposited into the pores of said carrier polymer framework and deposited onto the surface of said carrier polymer framework, wherein the carrier polymer framework is made of at least one polymer and the filler is a lipophilic biodegradable organic compound which is solid at room temperature and has a melting point above 40° C. The thickness of the membrane is between 20 and 200 nm and the microcapsule diameter is between 1 and 50 pm. Biodegradable microcapsules as described in the present invention are used in the form of aqueous dispersions as additives to fabric softeners, detergents, pesticides, pharmaceutical ingredients, paints, cosmetics products and the like.

The present invention relates to biodegradable microcapsules with a membrane wall formed from a biodegradable composite material and to the process of their synthesis. Biodegradable microcapsules of this invention are in a form of encapsulated particles in a water dispersion used for encapsulation of fragrances, active pharmaceutical ingredients, pesticides, and other materials that are subsequently used in fabric softeners, detergents, pesticides, paints, cosmetics and similar products.

### PRIOR ART

Microencapsulation is an established process in which an active ingredient is coated with a membrane, also known as a wall. The primary purpose of microencapsulation is to protect active ingredients inside the core from outside factors and prolonged or controlled release of these active ingredients. The end product of microencapsulation are microcapsules, composed of a core material, containing at least one active ingredient, and a wall. Typically, microcapsules range between 10⁻⁶ and 10⁻⁴ m in size.

Polyurea, polyacrylate, polyurethane and similar microcapsules are well known and widely used in numerous fields, notably in the pharmaceutical industry and the industry of fragrances and personal care products. The processes and technologies for the synthesis of microcapsules vary from field to field. The type of technology selected depends on the desired wall material, the properties of the core material and the end application. Microencapsulation techniques can generally be divided into chemical and physical techniques, depending on how the wall material is formed. Only a brief overview of microencapsulation techniques relevant to the invention are presented in this document, namely the synthesis of microcapsules from emulsions.

The commonly used method to prepare microcapsules from emulsions is interfacial polymerization. Firstly, a stable emulsion of two immiscible fluids is formed using surface active agents (surfactants) by dispersing one phase in the other. When a water and oil phase is used, water in oil (W/O) or oil in water (O/W) emulsions can be prepared. In this invention we will focus on O/W emulsions as the invention relates to the encapsulation of organic active compounds. The addition of monomers to each phase forming the final capsule membrane is characteristic of interfacial polymerization. By adding one type of monomer to each phase, the reaction between monomers takes place in the interfacial phase of emulsion droplets. After a stable emulsion is formed, the process of polymerization is initiated (temperature, pH, catalyst . . . ) leading to the formation of the final polymer and trapping each droplet in the membrane.

The above technique is frequently used to encapsulate fragrances as it allows for the slow release of the core material and consequently a long-lasting scent (US20150044262A1), while also protecting the fragrance from oxidation and rapid evaporation. In intensive agriculture, the same technique allows for the long-lasting effectiveness of pesticides and insecticides while also protecting them from UV-degradation (EP2403333A1, U.S. Pat. Nos. 5,160,529A, 4,956,129A). In the examples described, polyurea and melamine formaldehyde microcapsules are frequently used. In particular, the use of the latter is being increasingly limited as they contain traces of formaldehyde, which is toxic.

Other methods of microcapsule synthesis from emulsions are suspension polymerization and coacervation. In suspension polymerization the water (continuous) phase does not contain monomers, but rather a water-soluble initiator, which initiates polymerization at the interfacial interface. An example of suspension polymerization is the synthesis of polyacrylate microcapsules.

In coacervation, microcapsules are formed in colloidal systems with phase separation. One phase is rich in macromolecules (coacervate) whereas the other is poor in macromolecules. The two phases exist in equilibrium. Phase separation is prompted by a change in parameters such as pH and/or temperature or by the addition of a coagulant. This reduces the solvation shell, causing phase separation. The resulting coacervate then positions itself at the phase interface of emulsion droplets, encasing them in a membrane. This membrane can then be further chemically crosslinked.

It is important to note that microcapsules obtained using different techniques have different properties. Chemical encapsulation techniques such as interfacial and suspension polymerization allow for the formation of more resistant microcapsules as most polymers are inert. Microcapsules obtained in these ways also enable the preparation of capsules with lower porosity as they can be more crosslinked to any desired degree. Such capsules are preferably used with volatile compounds such as fragrances and etheric oils. In the case of physical and physical-chemical encapsulation techniques, such as coacervation, the membranes are not as crosslinked and as resistant because it is usually preferred for the wall material to slowly degrade and release the core material. These membranes consist of natural polymers such as polysaccharides and/or proteins. These encapsulation techniques are preferably used in the pharmaceutical and food industries, where microcapsules must be biocompatible and/or biodegradable. The biodegradable microcapsules described in this invention are primarily synthesized from emulsions.

The problem of non-degradable microplastic is rapidly gaining attention with the increasing focus on sustainable development. Microplastics (including microcapsules) made with crosslinked polymers, present in cosmetics and personal care products, are especially problematic as they get washed away into the sea. There they slowly degrade for centuries or, in the worst-case scenario, accumulate in wildlife.

Although biodegradable microcapsules from natural materials do exist, they are not suitable for certain applications. They generally do not encapsulate the (volatile) core material well enough and result in poorer mechanical properties and stability in different detergents and fabric softeners.

This problem is solved with microcapsules from a composite material based on the present invention. These capsules have good mechanical properties and are stable in basic detergents and fabric softeners over prolonged periods of time despite the low percentage of crosslinked material. The standard OECD 301 closed bottle biodegradability test in an enclosed respirometer measuring the uptake of oxygen has proved that microcapsules from composite material based on the present invention are biodegradable.

### DETAILED DESCRIPTION OF INVENTION

The present invention relates to the process of encapsulation of liquid organic components or solutions that do not mix with water. The present invention relates to the synthesis of the biodegradable microcapsule slurry as well as the biodegradable microcapsules themselves. Microcapsules prepared in accordance with the present invention are especially suitable for use in fabric softeners, detergents, personal care products and pharmaceuticals. The present invention is not limited to the above applications only and is suitable for the encapsulation of any active compound that allows for its encapsulation with the methods described in the present invention. The microencapsulation procedure described in the present invention allows for the encapsulation of a broad spectrum of liquid organic compounds (or solutions) into microcapsules made from a biodegradable composite material.

The present invention is described in more detail below and presented in figures as follows:

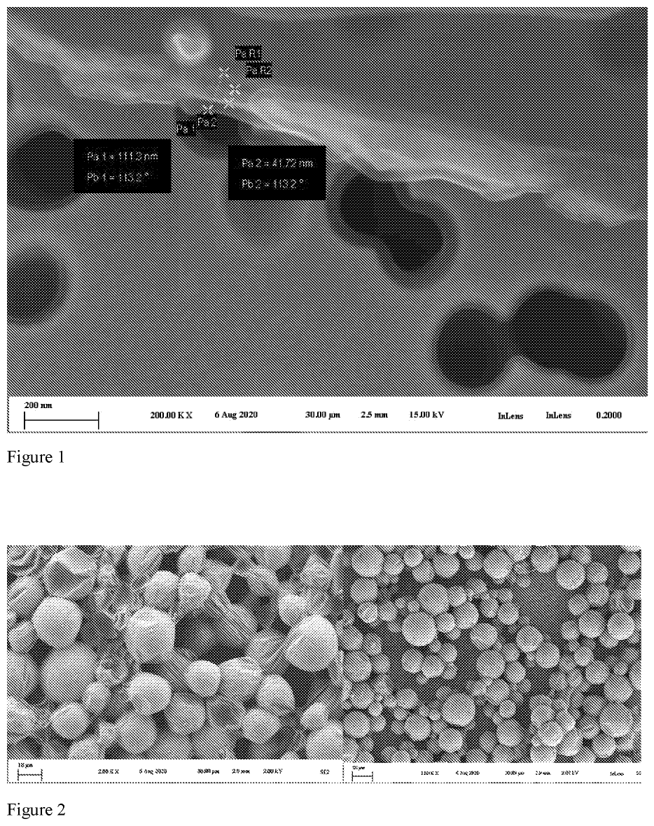

FIG. 1 shows a SEM image of a cross section of a biodegradable microcapsule obtained as described in the present invention.

FIG. 2 shows a comparison of a SEM image of biodegradable microcapsules obtained as described in the present invention (left) and classic polymer microcapsules (right).

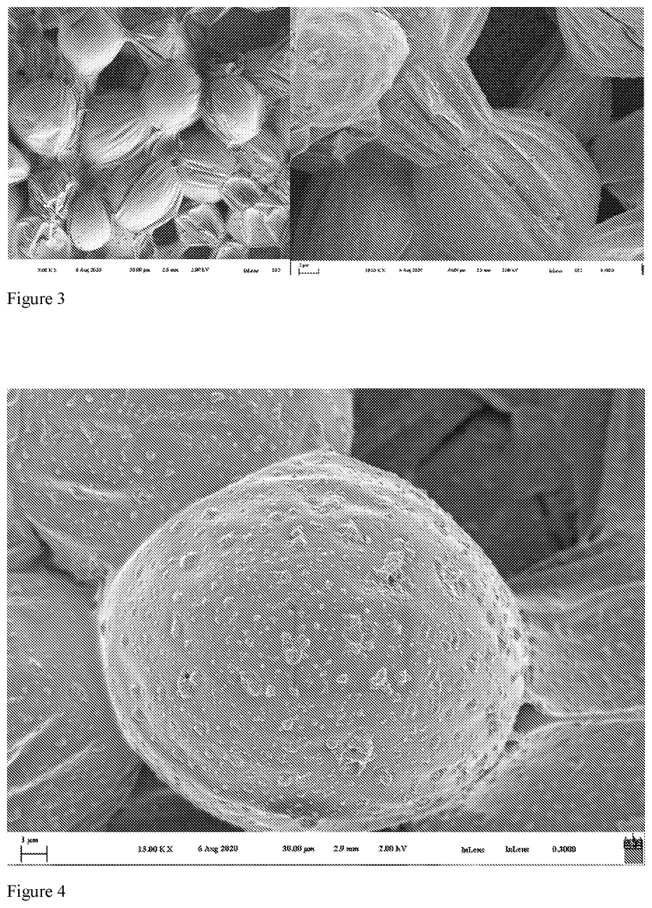

FIG. 3 shows a SEM image of the morphology of biodegradable microcapsules obtained as described in the present invention.

FIG. 4 shows a SEM image of pores in the membrane of biodegradable microcapsules obtained as described in the present invention that are filled in with filler.

FIG. 5 shows a SEM image of melted filler on the carrier polymer framework of the biodegradable microcapsule obtained as described in the present invention.

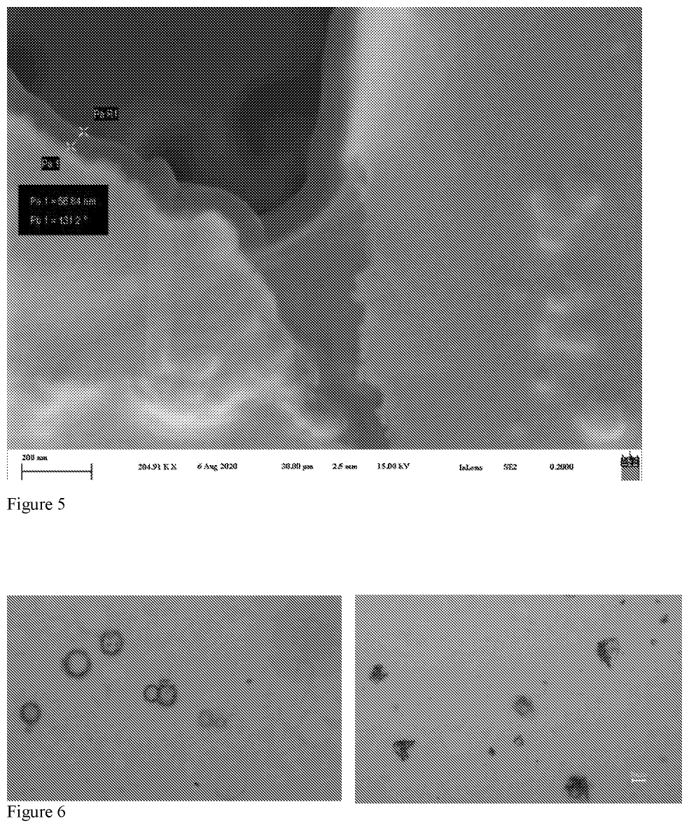

FIG. 6 shows biodegradable microcapsules with filler obtained as described in the present invention (left) and without filler (right) in a fabric softener base after 7 days.

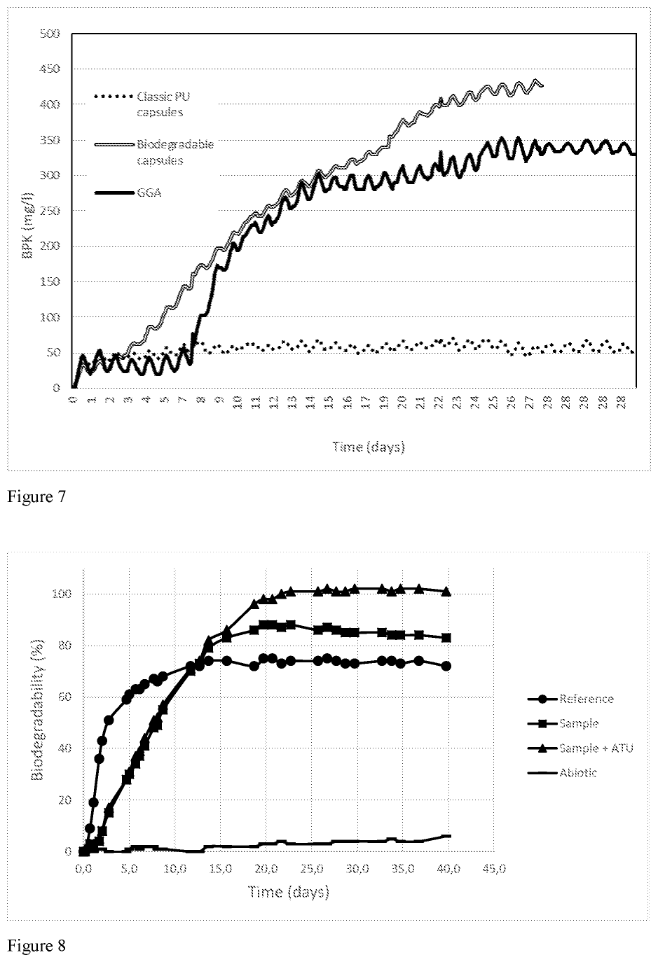

FIG. 7 shows the results of a quick respirometric biodegradability test.

FIG. 8 shows biodegradability results relative to time.

The biodegradable microcapsule of the present invention consists of
a core material, comprised of at least one liquid active component that does not mix with water and
a membrane, encapsulating the core material, wherein the membrane is comprised of a composite material comprising a carrier polymer framework and at least one filler embedded in the pores of this polymer framework and deposited on the surface of the carrier polymer framework, wherein said carrier polymer framework is comprised of at least one polymer, and the filler is a lipophilic biodegradable organic compound solid at room temperature and with a melting temperature above 40° C., wherein the thickness of the membrane is in the range of 20-200 nm and the diameter of the microcapsule is in the range of 1-50 μm.

The portion of the core material is 20-40% (w/w) of the end product that is a water dispersion (microcapsule slurry) and between 75% and 95% in a dry microcapsule.

The portion of the filler relative to the carrier polymer framework is between 5% and 95% (w/w), preferably in the range of 50-90% (w/w).

Given that the synthesis of biodegradable microcapsules as described in the present invention is carried out by interfacial polymerization in an emulsion, the active component to be encapsulated preferably has the following properties:
it does not mix with water and the log P (partition coefficient) values of all compounds present in the core material are above 2;
it allows a mixing of filler in the active component at elevated temperatures and is at the same time inert to the filler material (the active component should not react with the filler);
it allows a mixing of the reactants necessary for polymerization and is at the same time inert to these reactants (the active component should not react with the reactants needed for polymerization);
it is stable at temperatures up to 100° C. as the synthesis procedure takes place at an elevated temperature.

The suitable active components are selected from fragrances, pigments, insecticides, pharmaceutical ingredients, phase change materials, etheric oils (e.g. eucalyptus oil, lavender oil, rose oil, common valerian oil, basil oil, juniper oil, citronella, lemon grass oil, and others), other oils (e.g. palm oil, coconut oil, castor oil, sunflower oil, olive oil, mineral oil) and photochromic materials.

The active component in the microcapsule core can be present either alone or dissolved in an appropriate organic solvent. Appropriate organic solvents are immiscible with water with log P values above 2. The organic solvent should be compatible with the active component and with the reactants used, which means the organic solvent should not react with the active component and the used reactants.

Suitable polymers are selected from polyurea, polyurethane, polyacrylate, polyamide, polyester, and gelatin or other polymers suitable for polymerization in an emulsion.

When selecting an appropriate filler, its biodegradability and miscibility in different organic solvents are of utmost importance. The miscibility of the filler in a solvent should be such that it allows miscibility at higher temperatures (above 40° C.) but is immiscible at room temperature to allow for maximum filler crystallization when cooled in a controlled fashion to temperatures below 40° C. A suitable filler is chosen from waxes, paraffins, fatty acids and polyethylene glycols with solubility highly dependent on temperature. The most appropriate fillers are highly crystalline waxes with crystallization temperatures above 40° C.

The synthesis of a water dispersion of biodegradable microcapsules from emulsions as described in the present invention comprises the following steps:
a) preparation of an oil phase, wherein the core material to be encapsulated (the active ingredient and, if used, an organic solvent) is mixed with the filler and reactants suitable for forming a carrier polymer framework of the membrane at a temperature between 40° C. and 70° C., wherein the reactants are chemicals that mix with the core material and react during the polymerization phase to form the carrier polymer framework comprised of at least one polymer;
b) preparation of a water phase at a temperature higher than 40° C., which includes a water solution of biodegradable surface active ingredients;
c) preparation of a stable emulsion at a temperature between 40° C. and 70° C., wherein the oil phase is emulsified in the aqueous phase, forming dispersed or emulsified droplets the size of the microcapsules being formed;
d) formation of a carrier polymer framework of the membrane from at least one polymer, wherein water-soluble reactants are added to the stable emulsion, triggering the formation of a carrier polymer framework around the dispersed droplets at the phase boundary and thus the formation of an aqueous dispersion of microcapsules;
e) controlled cooling of the aqueous dispersion of microcapsules to a temperature between 10° C. and 25° C., whereby the filler crystallizes and is embedded into the pores and deposited onto the surface of the carrier polymer framework of the membrane and whereby a final aqueous dispersion of biodegradable microcapsules is formed with a mass fraction between 25% and 50%.

Optionally, the synthesis of the water dispersion of biodegradable microcapsules also includes a step f), where a stabilizer is added to the water dispersion of microcapsules to prevent the separation of microcapsules and water phase in the water dispersion, and/or additional reagents are added to ensure the end of polymerization and the elimination of surplus reactants, and/or pH regulators are added to set the pH value of the water dispersion to a desired value, mainly to better ensure the stability of the water dispersion or for easier use of the water dispersion in end products.

Step f) can follow step d), meaning the additions are added into the water dispersion prior to controlled cooling, or step f) can follow step e).

Choosing a filler with a different melting temperature directly affects its crystallization during controlled cooling. When the water dispersion is cooled below the melting temperature of the filler, the filler has a strong tendency to form crystals (it exhibits self-nucleating properties), leading to the eventual separation of the filler from the carrier oil phase. This property of the filler is utilized for the synthesis of biodegradable microcapsules. After the finished polymerization step and thus the formation of the polymer carrier framework, the water dispersion of biodegradable microcapsules is slowly cooled to temperatures between 10° C. and 25° C., causing the filler to separate from the core material and crystalize on the surface of the carrier framework, filling its pores and consequentially increasing the hardness and lowering the permeability of the membrane. The result of the synthesis is a stable 25-50% dispersion of microcapsules by mass of microcapsules.

Choosing the reactants added to the oil phase and water-soluble reactants added to the water phase depends on the choice of polymer to form the carrier polymer framework.

To form the carrier polymer framework from polyurea and polyurethanes, suitable reactants are chosen amongst isocyanates, especially aromatic and aliphatic isocyanates with at least two functional groups. Reactants are chosen predominantly amongst aromatic and aliphatic isocyanates and include toluene diisocyanate (TDI), hexamethylene diisocyanate (HDI), isophorone diisocyanate (IPDI), methylene diphenyl diisocyanate (MDI), and their oligomers. Suitable water-soluble reactants include polyols and amines. Suitable polyols are polyols with at least two functional groups, e. g. ethylene glycols, pentaerythritol, sorbitol, butanediol, hexanediol, pentanediol and caprolactone diols. Suitable amines include, but are not limited to, diethylenetriamine, ethylenediamine, melamine, hexamethylenediamine, chitosan, gelatin, polyethyleneimines and guanidine.

To form the carrier polymer framework from polyacrylates, suitable reactants are chosen amongst acrylates and initiators. Reactants are chosen predominantly amongst multifunctional acrylates and methacrylates, e.g. allyl methacrylates, dimethacrylates, diacrylates and butylaminoethyl methacrylate. Suitable water-soluble reactants are initiators such as peroxy initiators, for example benzoyl peroxide and ammonium persulphate.

To form the carrier polymer framework from polyamides and polyesters suitable reactants are chosen from acyl dichlorides. Reactants are chosen predominantly from dichlorides such as sebacoyl dichloride, adipoyl dichloride and benzenesulfonyl dichloride. Suitable water-soluble reactants are diols and polyols for the synthesis of polyesters (same as described above) and diamines and polyamines for the synthesis of polyamides (same as described above).

Reactants suitable for forming the carrier polymer framework using coacervation are gelatin and carboxymethylcellulose and other suitable polymers, e. g. chitosan and ethylcellulose. Suitable water-soluble reactants include, but are not limited to, glutaraldehyde, carbodiimide and glyoxal.

Surface active agents prevent droplet coalescence when preparing an emulsion, resulting in a stable emulsion. Suitable surface active agents are chosen amongst anionic, cationic and nonionic emulsifiers and stabilizers. Suitable anionic emulsifiers are sulphates, sulfonates, phosphates, and carboxylates, e.g. sodium lauryl sulphate, sodium dodecyl sulphate, sodium stearate and acrylates. Suitable cationic emulsifiers include, but are not limited to, quaternary ammonium salts. Suitable nonionic emulsifiers are all emulsifiers with an HLB value above 7. Additionally, stabilizers dissolved in the water phase, acting as steric barriers preventing oil droplet coalescence, can be used. Suitable stabilizers include, but are not limited to, carboxymethylcellulose, polyvinyl alcohols, polysorbates, polyethyleneimines, gum Arabic, glycerol monostearate and similar.

A dedicated homogenizer and/or a mechanic stirrer at high revolutions is used for the preparation of the emulsion.

At the phase interface, where the reactants in the oil phase come into contact with the reactants in the water phase, a thin polymer layer is formed, entrapping the filler material and serving as a carrier framework for the subsequent deposition of filler into the pores between the polymer chains of the carrier polymer framework and on the surface of the carrier polymer framework.

Optionally, depending on the choice of polymer, a catalyst can be added during the formation of the polymer framework of the membrane (during the polymerization step). For example, when the carrier framework is formed from poly(urea-urethanes), with diisocyanates and polycaprolactone polyols in the oil phase and water-soluble polyols and polyfunctional amines in the water phase, bismuth neodecanoate or DABCO is used as catalyst. At lower temperatures diisocyanates only react with amines, but with the addition of a catalyst and when heating the reaction mixture to 80° C. diisocyanates also react with polyols to form a carrier polymer framework made from poly(urea-urethanes).

Polymerization takes place at an elevated temperature for up to 150 minutes, and the reaction is monitored using IR spectroscopy.

The use of filler for the preparation of the oil phase (i.e. mixing filler with core material with at least one active component) affects the process of polymerization. With some active components, especially with some fragrances and etheric oils, polymerization is hindered or prevented entirely because of the interaction between the reactants and the core material which reduces the usability of the process of encapsulation. The result of microencapsulation could be drastically improved by adding filler to these problematic active components. The filler has good emulsion-stabilization properties and positively impacts encapsulation itself as it effectively dilutes the core material and increases the diffusion of reactants to the phase interface of emulsion droplets where the carrier polymer framework is formed. Aside from biodegradability, stability and fragility of the microcapsules, the filler also adds to the robustness of the encapsulation method described in present invention.

FIG. 1 shows a cross-section of a microcapsule obtained as described in the present invention. The inner diameter R2 represents the thickness of the polymer layer (carrier polymer framework), while the outer diameter R1 represents the thickness of the entire microcapsule membrane, including both the carrier polymer framework and the deposited filler layer. The membrane thickness in this example is between 110 nm and 120 nm.

FIG. 2 shows the capsule surface of the classic polymer membrane microcapsule (right) and the biodegradable microcapsule obtained as described in the present invention (left). The difference in surface appearance is highly noticeable. Classic microcapsules are very robust and inert, whereas biodegradable microcapsules are entirely covered with a filler, proving the filler is present on the entire microcapsule surface and further proving the non-separable nature of the composite membrane consisting of a carrier polymer framework completely covered with the filler. It is also evident from FIG. 2 that the filler crystallizes and is thus largely removed from the oil phase and is not present in the oil phase in larger quantities and that it also does not react with the active component in any way.

FIG. 3 shows the morphology of microcapsules obtained as described in the present invention. The filler covering the entire microcapsule surface is clearly visible. Because of the filler's presence on the surface, microcapsules are also fused together into larger agglomerates with the filler as binder.

FIG. 4 shows the pores of biodegradable microcapsules obtained as described in the present invention. It is evident that the filler is deposited in greater amounts where pores are present in the microcapsule surface.

By focusing the electron beam onto a crack in the microcapsule membrane, the material is heated to a temperature high enough to melt the crystalized filler. The filler in its liquid state is permeable to electrons, which allows for the revelation and identification of the carrier polymer framework underneath the layer of crystallized filler. This proves that the filler is deposited into the pores of the carrier polymer framework and that it is also deposited onto the surface of a microcapsule (FIG. 5). It is also possible to roughly estimate the thickness of the filler layer, which is 50-60 nm in the given example.

The described procedure allows the synthesis of a range of biodegradable microcapsules with different properties, dependent on the membrane composition and the type and quantity of the filler used. The type of membrane and the type and quantity of filler used depends on the end use of the product. In the case of fragrance encapsulation for use in fabric softeners and detergents, fragile and highly impermeable microcapsules are favored to ensure the release of the fragrance only upon the rubbing of textile. With classic microcapsules, this effect is achieved with a thicker and more chemically cross-linked membrane. The present invention allows the synthesis of biodegradable microcapsules with a thinner membrane as the fragility and impermeability of the membrane is also warranted by the filler and not solely by the carrier polymer framework. With microcapsules obtained as described in the present invention, the membrane also has a lower degree of crosslinking (between 0% and 20%) and thus a higher degree of biodegradability. Use of the filler allows for crosslinking to be reduced or even eliminated. In the case of 0% cross-linking, the degree of biodegradability is 100% when using bio polymers.

### Examples

### 1. Synthesis of Biodegradable Microcapsules in a Water Dispersion with a Poly(Urea-Urethane) Polymer Carrier Framework

300 g water, 6 g polyvinyl alcohol (PVA) (Celvol 205) and 3.6 g carboxymethylcellulose (CMC) (Carbofix 5A) are mixed in a reactor, heated to 80° C. and mixed for 1 hour at 80° C. for PVA and CMC to completely dissolve. The mix is then cooled to 40° C. Separately, 150 g fragrance (from a different manufacturer) is heated up to 40° C. in a beaker. 20 g wax with a melting point of 44° C., 1.5 g toluene diisocyanate (TDI), 1.5 g polyisocyanate Desmodur N3400 and 1 g CAPA 3031 are then added to the fragrance and mixed thoroughly. The obtained mixture is then poured into the reactor, and the mixing speed in the reactor is increased. This new mixture is then mixed until emulsion droplets of the desired size between 10 and 40 μm are obtained. Once the proper emulsion is obtained, 10 g of a mixture of 5 g cationic surfactant (Lupasol PS) and 5 g 10% water solution (w/w) of diethylenetriamine is added. The mixture is left to stir for 10 min after which 10 g 50% water solution (w/w) of xylitol/sorbitol/maltodextrin is added. Immediately after that, the catalyst BorchiKat 315 is added and the mixture is then heated to 80° C. and maintained at this temperature for 2.5 h. After this time, the mix is cooled to room temperature. The end product produced in this way is a water dispersion of microcapsules with the following content: 63% (w/w) water solution of emulsifiers and stabilizers and 37% (w/w) microcapsules with the core material and the membrane. 30% (w/w) of end product is represented by the fragrance, whereas the fragrance represents 84% (w/w) of dry microcapsule weight. The ratio of the capsule membrane is 16% (w/w) of dry microcapsule weight.

### 2. Synthesis of Biodegradable Microcapsules in a Water Dispersion with a Poly(Urea-Urethane) Polymer Carrier Framework with a Different Surfactant Ratio

230 g water, 6 g polyvinyl alcohol (PVA) (Celvol 205) and 3.6 g carboxymethylcellulose (CMC) (Carbofix 5A) are mixed in a reactor, heated to 80° C. and mixed for 1 hour at 80° C. for PVA and CMC to completely dissolve. The mix is then cooled to 40° C. Separately, 150 g fragrance (from a different manufacturer) is heated to 40° C. in a beaker. 20 g paraffin wax with a melting point of 44° C., 2.5 g toluene diisocyanate (TDI), and 2.5 g polyisocyanate Desmodur N3400 are then added to the fragrance and mixed thoroughly. The obtained mixture is then poured into the reactor and the mixing speed in the reactor is increased. This new mixture is then mixed until emulsion droplets of the desired size between 10 and 40 μm are obtained. Once the proper emulsion is obtained, 10 g of a mixture of 4 g cationic surfactant (Lupasol PS) and 15 g 10% water solution (w/w) of diethylenetriamine is added. The mixture is left to stir for 10 min after which 8 g 50% water solution (w/w) of sorbitol is added. Immediately after that, the catalyst BorchiKat 315 is added and the mixture is then heated to 80° C. and maintained at this temperature for 2.5 h. After this time the mix is cooled to room temperature. The end product produced in this way is a water dispersion of microcapsules with the following content: 56% (w/w) water solution of emulsifiers and stabilizers and 44% (w/w) microcapsules with the core material and the membrane. 34% (w/w) of the end product is represented by the fragrance, where the fragrance represents 84% (w/w) of dry microcapsule weight. The ratio of the capsule membrane is 16% (w/w) of dry microcapsule weight.

### 3. Synthesis of Biodegradable Microcapsules in a Water Dispersion with a Poly(Urea-Urethane) Polymer Carrier Framework for Use in Detergents

309.6 g water, 6 g polyvinyl alcohol (PVA) (Celvol 205) and 3.6 g carboxymethylcellulose (CMC) (Carbofix 5A) are mixed in a reactor, heated to 80° C. and mixed for 1 hour at 80° C. for PVA and CMC to completely dissolve. The mix is then cooled to 40° C. Separately, 150 g fragrance (different manufacturers) is heated to 40° C. in a beaker. 30 g paraffin wax with a melting point of 42° C., 2.5 g toluene diisocyanate (TDI) and 1.5 g polyisocyanate Desmodur N3400 are then added to the fragrance and mixed thoroughly. The obtained mixture is then poured into the reactor and the mixing speed in the reactor is increased. This new mixture is then mixed until emulsion droplets of the desired size between 10 and 20 μm are obtained. Once the proper emulsion is obtained, 5 g of 20% water solution (w/w) of diethylenetriamine is added. The mixture is left to stir for 10 min after which 1.5 g of dry pentaerythritol is added. Immediately after that, the catalyst BorchiKat 315 is added and the mixture is then heated to 80° C. and maintained at this temperature for 1 h. After 1 hour, 3 g of xylitol/sorbitol/maltodextrin is added and the mixture is stirred at 80° C. for an additional time of 1 hour. After this time the mix is cooled to room temperature. The end product produced in this way is a water dispersion of microcapsules with the following content: 65% (w/w) water solution of emulsifiers and stabilizers and 35% (w/w) microcapsules, counting the core material and the membrane. 30% (w/w) of the end product is represented by the fragrance, where the fragrance represents 84% (w/w) of dry microcapsule weight. The ratio of the capsule membrane is 16% (w/w) of dry microcapsule weight.

### 4. Synthesis of Biodegradable Microcapsules in a Water Dispersion with a Gelatine-Based Polymer Carrier Framework and a 21% (w/w) Share of Core Material

300 g water and 12.4 g gelatine are mixed in a reactor, heated to 50° C. and mixed until gelatine is completely dissolved. Separately, 150 g fragrance (from a different manufacturer) is heated to 50° C. in a beaker, to which 10 g paraffin wax with a melting point of 44 g is added and mixed thoroughly. The obtained mixture is then poured into the reactor and the mixing speed in the reactor is increased and, if necessary, a specialized homogenization tool is used. This new mixture is then mixed until emulsion droplets of the desired size between 10 μm and 20 μm are obtained. Once the proper emulsion is obtained, 8 g of carboxymethylcellulose dissolved in 136 g water is added. After being thoroughly mixed, 10% (w/w) solution of acetic acid is added dropwise until a pH value of 4.0 is reached. The mixture is then slowly cooled to a temperature of 10° C. over the course of 1 h and after 15 minutes at 1° C. heated to 20° C. 1.9 g 50% (w/w) water solution of glutaraldehyde is added dropwise and the mixture mixed for a further 45 minutes. The end product produced in this way is a water dispersion of microcapsules with the following content: 74% (w/w) water and 26% (w/w) microcapsules, counting the core material and the membrane. 21% (w/w) of the end product is represented by fragrance, where the fragrance represents 88% (w/w) of dry microcapsule weight. The ratio of the capsule membrane is 12% (w/w) of dry microcapsule weight.

Biodegradable microcapsules obtained by procedures described in the present invention retain all the key properties of classic microcapsules. Properties such as end product (e. g. fabric softeners) stability and the successfulness of active component encapsulation do not change significantly compared to classic microcapsules, but microcapsules obtained by the procedures described in the present invention are biodegradable.

Analyses of Microcapsules

Procedure to Determine Microcapsule Stability in Fabric Softeners:

The microcapsule dispersion is mixed with the standard fabric softener base (1% w/w) and stored at 40° C. to simulate aging at an accelerated rate. The fabric softener sample obtained in this way is examined under a microscope on the day of the preparation and then every T in day for the following 28 days. Depending on the microscopic image, the sample is given a numerical grade representing microcapsule stability, where a grade of 5 means the capsules have retained the core material well, have no changes and are not in any way visibly damaged, while a grade of 1 means the microcapsules are entirely empty with their core material completely gone and are visibly damaged/destroyed.

The results of such analysis are given in Table 1. Samples 1 to 4 refer to the microcapsules prepared as described in the present invention in the execution examples section. For classic polymer capsules, the membrane consisted of only the carrier framework made from crosslinked poly(urea-urethane).

TABLE 1

Stability grade

| Sample | Day 1 | Day 7 | Day 14 | 1 month |
|---|---|---|---|---|
| Classic | 5 | 5 | 4 | 3 |
| 1 | 5 | 5 | 5 | 4 |
| 2 | 5 | 5 | 4 | 4 |
| 3 | 5 | 4 | 4 | 4 |
| 4 | 5 | 4 | 3 | 3 |

As evident from the results in Table 1, the biodegradable microcapsules obtained as described in the present invention have a high degree of stability, comparable with classic microcapsules.

The most important indicator of quality of encapsulation is the end application. To evaluate this parameter, a scratch and sniff technique is used where a water dispersion of microcapsules is applied to a target surface and dried thoroughly. In case of a successful fragrance encapsulation, an intensive scent is released upon rubbing the surface.

To determine the quality of microcapsules regarding fragrance intensity upon rubbing an odorless fabric, a softener base with an added 1% (w/w) microcapsule dispersion was used. Cotton towels were washed in a washing machine at 40° C. and this prepared sample was used as fabric softener. After the towels were thoroughly dried, scent intensity was graded on a scale of 1 to 5 upon rubbing the towel, with 5 being the highest grade (most intensescent).

The results are given in Table 2. Samples 1 to 4 refer to the microcapsules prepared as described in the present invention in the execution examples section. For classic polymer capsules, the membrane consisted only of the carrier framework made from crosslinked poly(urea-urethane).

TABLE 2

| Sample | Intensity |
|---|---|
| Classic | 5 |
| 1 | 5 |
| 2 | 5 |
| 3 | 3 |
| 4 | 3 |

As evident from the results in Table 1, the biodegradable microcapsules obtained as described in the present invention have a high degree of stability, comparable with classic microcapsules.

To further confirm that microcapsules obtained as described in the present invention retain the core material as well as classic microcapsules despite a thinner polymer membrane, we have synthesized two types of microcapsules with comparable membrane thickness, namely classic polymer microcapsules with a high degree of crosslinking and biodegradable microcapsules as described in the present invention. With the classic polymer microcapsules, the membrane consisted only of a polymer network, namely a crosslinked poly(urea-urethane) network, with the membrane thickness between 100 and 150 nm, whereas microcapsules obtained as described in execution example 1 in the present invention had a membrane wall thickness of 110-120 nm. Gravimetric analysis at 50° C. reveals that the mass of the sample of biodegradable microcapsules obtained as described in the present invention is dropping much more slowly and levels off sooner as opposed to classic microcapsules without filler material. This confirms that the membrane obtained as described in the present invention is less porous and thus less permeable to fragrances.

These results were further confirmed by stability tests in fabric softeners. Classic microcapsules without filler material are much worse at retaining the core material and are completely emptied already after 7 days (FIG. 6), whereas biodegradable microcapsules obtained as described in the present invention contain a large portion of the core material even after 28 days.

Quick Respirometry Biodegradability Tests

Comparable respirometric tests in reagent bottles per standard method 5210 BIOCHEMICAL OXYGEN DEMAND (BOD) (2017) were carried out. These tests were carried out in a mineral substrate: 250 mL deionized water with the following nutrients added:

1. BPK1 (Phosphate Buffer)
KH2PO4 8.5 mg/l
K2HPO4 21.75 mg/l
Na2HPO4×7H2O 33.4 mg/l
NH4C1 1.7 mg/l

2. BPK2 (Magnesium Sulfate)
MgSO4×7H2O 22.5 mg/l

3. BPK3 (Calcium Chloride)
CaCl2 27.5 mg/l

4. BPK4 (Iron Chloride)
FeC13×6H2O 0.25 mg/l

In a classic respirometric test a GGA (300 mg/l) mix of glucose and glutamic acid is used as a positive control sample. A mix of bacterial culture from the laboratory was used as the inoculum.

Description of the Test Method

Microcapsules obtained as described in the present invention were first mechanically damaged using a planetary ball mill, the fragrance then evaporated, and the resulting dry sample dispersed in clean water. The amount of microcapsule dispersion was calculated so that the volume of the added end dispersion of membrane material amounted to 100 mg of membrane material:
Microcapsule dispersion obtained as described in execution example 1 in the present invention: 0.12 mL
Inoculum: 10 mL
Distilled water: 238.88 mL
BPK: 0.25 μL.

It is evident from the results of quick respirometric biodegradability tests (FIG. 7) that microcapsules with biodegradable filler material (composite membrane) obtained as described in the present invention degrade much faster in comparison to classic polymer microcapsules. The rate of biodegradability for the former is higher than that of the positive control sample with glucose (GGA).

OECD 301 Closed Bottle Biodegradability Test

To confirm the biodegradability of the microcapsule sample obtained as described in execution sample 1 in the present invention, a biodegradability test following OECD 301 F (Determination of ready biodegradability in a closed respirometer by measuring oxygen consumption) was carried out. The test was placed on the list of suitable methods for testing biodegradability of microplastics by the European Chemicals Agency (ECHA).

a) Description of the Test Method

Microcapsule sample 1 was prepared by filtering the water dispersion of microcapsules and rinsing it with water to remove water-soluble components (emulsifiers, non-reacted reactants). The sample obtained in this way was then dried at 80° C. to remove the fragrance from the microcapsule core. The dry solid remains of the sample consisted only of the microcapsule membrane and served as the sample to be used in the biodegradability test.

For the test, we used activated sludge from a municipal wastewater treatment plant. The sludge was collected the day before the biodegradability test, washed at least 5 times with tap water, and its concentration (mg MLVSS/L) was determined by filtering 20 mL of a suspension of activated sludge with black ribbon filter paper. The sludge was then placed in a climate chamber (22±2° C.), where it was stirred and aerated until it was used.

The performed biodegradability assessment test is one of the optional tests for determination of ready biodegradability. It is based on the measurement of oxygen consumption in a closed respirometer, where biodegradation is measured indirectly through oxygen consumption at a constant temperature of 20±1° C. for 40 days. The concentration of activated sludge in the test was 30 mg/L. It was not necessary to adjust the pH before the test because the pH of the test mixture was 7.8±0.0. (The optimal range is between 6-8.) In parallel with that, we also performed a test with a reference substance (sodium acetate), which confirmed the activity of microorganisms and regular conditions for biodegradation throughout the test. Abiotic degradation was also determined in a system without the addition of activated sludge to the mixture, at the same time chemically sterilized by adding HgCl₂. The concentration of the sample in the test was 0.18 vol. % (COD=100 mg/L). Abiotic degradation was also measured with the same sample concentration. Each test was performed in parallel. The test with the same sample concentration and with added allylthiourea—ATU (4 mL/L) as a nitrification inhibitor was also performed. Thus, the measured oxygen consumption was proven to be due to the (bio) degradation of the sample and not to nitrification.

b) Test Results

Average chemical oxygen demand (COD) was determined. pH of the microcapsule sample was 7.5±0.1.

| Sample repetition | COD (mg/L) |
|---|---|
| 1 | 53.925 |
| 2 | 63.481 |
| 3 | 57.318 |
| 4 | 50.076 |
| Average | 56.200 |

In parallel with that, we also measured oxygen consumption in the blank sample, in the test with the reference substance (sodium acetate), in the sample and in the abiotic sample. We also checked oxygen consumption in the blank sample and the sample with added ATU to make sure that nitrification (oxidation of ammonium, which is not (bio) degradation of the sample) and consequent oxygen consumption, does not occur. The initial pH of the mixture was in the range of 7.7±0.1, so according to the standard ISO procedure the pH regulation was not necessary and the pH of the sample had no effect on biodegradation.

The results showed (FIG. 8) that the reference compound degraded well. More than 60% was degraded after only 5 days. This confirmed the activity of microorganisms, the adequacy of the test and the validity of the results. The sample also degraded well in the test. In the 40 days of the test more than 80% degradation was achieved (85±3%). This level of degradation was reached on the 17^{th} day of the test, after a 3-day lag phase. The rapid degradation of the sample was also confirmed in the test with the sample with added ATU (added to prevent nitrification and consequent oxygen consumption), as in this case complete degradation of the sample (99±2%) was achieved. Since the degradation in the sample with ATU is comparable to and even higher than that in the sample without ATU added, it can be concluded that the difference between the two curves is due to experimental error or rather due to the principle of the test, and nitrification did not occur. It means that the actual biodegradation of the sample reached up to 85% with minimal abiotic degradation (6±0%). It can be concluded that the attained level of degradation is mainly result of microorganism activity (biodegradation).

1. A biodegradable microcapsule based on a composite material characterized in that the microcapsule is comprised of:
a core material, comprised of at least one liquid active component that does not mix with water and
a membrane, encapsulating the core material, wherein the membrane is comprised of a composite material comprising a carrier polymer framework and at least one filler embedded in the pores of said polymer framework and deposited on the surface of said carrier polymer framework, wherein said carrier polymer framework is comprised of at least one polymer and the filler is a lipophilic biodegradable organic compound that is solid at room temperature and has a melting temperature above 40° C., wherein a diameter of the microcapsule is in the range of 1-50 μm and a thickness of the membrane is in the range of 20-200 nm.

2. A biodegradable microcapsule according to claim 1, wherein the filler represents 5 to 95%, preferably 50 to 90% by weight relative to the weight of the polymer.

3. A biodegradable microcapsule according to claim 1, wherein the active component is selected from fragrances, pigments, insecticides, pharmaceutical ingredients, phase change materials, etheric oils (including, but not limited to, eucalyptus oil, lavender oil, rose oil, common valerian oil, basil oil, juniper oil, citronella, lemon grass oil and others) and other oils (including, but not limited to palm oil, coconut oil, castor oil, sunflower oil, olive oil and mineral oil) and photochromic materials.

4. A biodegradable microcapsule according to claim 1, wherein the active component in the core material is present either as a pure compound or dissolved in an organic solvent where the organic solvent is immiscible with water and does not chemically react with the active ingredient or the used reactants.

5. Biodegradable microcapsules according to claim 1, wherein the polymer is selected from polyureas, polyurethanes, polyacrylates, polyamides, polyesters and gelatin or other polymers suitable for polymerization from an emulsion.

6. A biodegradable microcapsule according to claim 1, wherein fillers are selected from waxes, paraffins, fatty acids and polyethylene glycols with high solubility temperature dependency.

7. A biodegradable microcapsule according to claim 6, wherein fillers are preferably highly crystalline waxes with a melting temperature above 40° C.

8. A synthesis process of water dispersion of biodegradable microcapsules according to claim 1 from emulsions comprising the following steps:
a) preparation of an oil phase, wherein a core material to be encapsulated is mixed with a filler and reactants suitable for forming a carrier polymer framework of a membrane at a temperature between 40° C. and 70° C., wherein the reactants are chemicals that mix with the core material and react during the polymerization phase to form the carrier polymer framework comprised of at least one polymer;
b) preparation of a water phase at a temperature higher than 40° C., which includes a water solution of biodegradable surface active ingredients;
c) preparation of a stable emulsion at a temperature between 40° C. and 70° C., wherein the oil phase is emulsified in the aqueous phase, forming dispersed or emulsified droplets the size of the microcapsules being formed;
d) formation of a carrier polymer framework of a membrane from at least one polymer, wherein water-soluble reactants are added to the stable emulsion, triggering the formation of a carrier polymer framework around the dispersed droplets at the phase boundary, and thus the formation of an aqueous dispersion of microcapsules;
e) controlled cooling of the aqueous dispersion of microcapsules to a temperature between 10° C. and 25° C., whereby the filler crystallizes and is embedded into the pores and deposited onto the surface of the carrier polymer framework membrane, and whereby a final aqueous dispersion of biodegradable microcapsules is formed with a mass fraction between 25% and 50%.

9. A synthesis process according to claim 8, wherein the process optionally includes a step f), where a stabilizer is added to the aqueous dispersion of microcapsules for preventing the separation of microcapsules and the water phase in the water dispersion, and/or the addition of residual reactants for completing the polymerization or eliminating the excess reactants, and/or pH regulators, wherein step f) follows step d), meaning that these additives are added into the water dispersion prior to controlled cooling, or step f) follows step e).

10. A synthesis process according to claim 8, wherein the reactants for the formation of the carrier polymer framework from polyureas and polyurethanes are selected from aromatic or aliphatic isocyanates with at least two functional groups, and the water-soluble reactants are selected from polyols with at least two functional groups and amines.

11. A synthesis process according to claim 8, wherein the reactants for the formation of the carrier polymer framework from polyacrylates are selected from multifunctional acrylates and methacrylates, and the water-soluble reactants are peroxy initiators, e.g. benzoyl peroxide and ammonium persulphate.

12. A synthesis process according to claim 8, wherein the reactants for the formation of the carrier polymer framework from polyamides or polyesters are selected from acidic dichlorides, and the water-soluble reactants are selected from diols or polyols for the synthesis of polyesters, and diamines and polyamines for the synthesis of polyamides.

13. A synthesis process according to claim 8, wherein the reactants for the formation of the carrier polymer framework by coacervation are selected from gelatin, chitosan or ethylcellulose, and the water-soluble reactants are selected from glutaraldehyde, carbodiimide or glyoxal.

14. A synthesis process according to claim 8, wherein the surface-active agents are selected from anionic, cationic, and nonionic emulsifiers and stabilizers, wherein the anionic emulsifiers are selected from sulphates, sulfonates, phosphates, and carboxylates, the cationic emulsifiers are quaternary ammonium salts, the nonionic emulsifiers are all emulsifiers with an HLB value above 7, the stabilizers are selected from carboxymethylcellulose, polyvinyl alcohols, polysorbates, polyethyleneimines, gum Arabic and glycerol monostearate.

15. A synthesis process according to claim 8, wherein optionally, depending on the choice of the polymer forming the carrier polymer framework, a catalyst is added in the polymerization phase when the carrier polymer frame is being formed.
